(19) United States
(12) Patent Application Publication
(10) Pub. No.: US 2010/0168563 A1
(43) Pub. Date: Jul. 1, 2010

(54) DEVICE AND METHOD FOR THE PROLONGED DELIVERY OF AN ACTIVE AGENT TO A BODY CAVITY

(76) Inventor: David Braver, Jerusalem (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 12/279,362

(22) PCT Filed: Feb. 15, 2007
(86) PCT No.: PCT/IL07/00217

§ 371 Date: Aug. 14, 2008

(30) Foreign Application Priority Data
Feb. 16, 2006 (IL) 173762

(51) Int. Cl. A61M 31/00; A61K 9/02; A61B 6/00

(52) U.S. Cl. 600/431; 604/285; 604/288; 604/514

(57) ABSTRACT

A device for application to a body cavity. The device is insertable into the cavity of a subject in need. The device includes a non-absorbable, flexible tube of an elongated shape, a removable core element situation within the tube, and a retention mechanism for maintaining the device within the cavity.

### FIELD OF THE INVENTION

The present invention relates in general to a device and method for the prolonged delivery of an active agent to a body cavity. The delivery may be for local or systemic treatment.

### BACKGROUND OF THE INVENTION

Publications and other reference materials referred to herein, including references cited therein, are incorporated herein by reference in their entirety and are numerically referenced in the following text and respectively grouped in the appended Bibliography which immediately precedes the claims.

There are many situations in which it is necessary for an active agent to be delivered to a body cavity. One of the best examples illustrative of such a situation is from the field of rectal disorders, specifically anal fissure.

Although it is not widely known, one of the most common reasons that someone visits a medical doctor is for the treatment of a rectal disorder. One example of a common disorder, which can affect men and women, both young and old, is anal fissure (1,2). Anal fissure (hereafter, “AF”) is a small split or tear in the mucosa that may cause painful bowel movements and bleeding. AF is extremely common in young infants but may occur at any age. Studies suggest that 80% of infants will have had AF by age one year. Most fissures heal on their own or as a result of minimal treatment; however, some fissures may require more serious medical, treatment.

The occurrence of AF decreases rapidly with age. Fissures are much less common among school-aged children than among infants. In adults, fissures may be caused by constipation, particularly when passing large, hard stools, or by prolonged diarrhea. In older adults, AF may be caused by decreased blood flow to the area (3). AF is also common in women after childbirth and in people with Crohn's disease.

The pain involved with AF causes both physical and emotional stress (4,5). This, in turn, causes the anus to contract during bowel movements, thereby aggravating the area even more. In some cases, one may be required to stay home, or even be hospitalized as a result of the AF.

Of those whose AF has been successfully treated by conventional methods, recurrence generally occurs between 30-70% of the time.(2) The following are several of the conventional methods of treating acute and chronic AF.

The simplest method of treating acute AF comprises warm baths and a diet rich in fiber. Approximately 80% of sufferers of acute AF are cured by following this approach, within a time span of three weeks. Use of additional means such as steroid creams, anesthetics, dilators, etc. will not help to quicken the speed of recovery, and it is best to not even use them (2).

Alternatively, to cure acute AF one might use a nitric oxide cream to rub around the anus. This cream relaxes the internal anal sphincter. It is reported that 92% of individuals who use nitric oxide cream are cured within only two weeks (6).

None of the above-mentioned techniques will lead to the healing of an individual suffering from chronic AF. All of the methods for treating chronic AF attempt to lower the anal pressure. One method involves performing an internal sphincterotomy (IS). The success rate of healing for those who undergo IS is extremely high. However, such a procedure results in a decrease in fecal continence of the individual, and, therefore, is not always recommended. Moreover, as with all surgical procedures, some health risks are involved, especially when dealing with a septic region like the anus. Furthermore, post-surgery pains are expected (2,7).

Anal dilation is another method of treatment of chronic AF. This is a crude and painful technique, wherein a series of increasingly larger diameter tubes are inserted to the rectum to enlarge the sphincter opening. There are several drawbacks to this method, including lack of control over the final size of the opening, and the chances of incontinence are greater than those following IS. Also, the success rate of anal dilation is relatively low (2).

Topical nitroglycerin (TN) is a cream that is used for treating chronic AF. The claimed success rate is between 68-85% within eight weeks. Drawbacks include reports of headaches by 58% of treated patients, and a high chance of relapse. Advantages include no reports of incontinence (7).

Another method of treating chronic AF is the injection of butulinum toxin (BT) into the internal and external sphincters of an individual, causing paralysis of the sphincters. This is not a surgical procedure, however, it is invasive. Although in the short term, the success rate is typically 80%, after an extended period of time, relapse is common. Relative to TN, use of BT can be expensive (2).

For individuals who regularly experience fecal incontinence—for example, the elderly, diabetics, those having bowel disorders, multiparous women, and patients with relapses after IS—plastic surgery, involving advancement of the anal flap might be recommended. However, similar to IS, this procedure is risky, and several drawbacks, particularly due to the region in which the procedure is performed, are associated with it.(8)

Of all of the above-mentioned methods of treatment of AF, none result in a complete healing of AF, and all have drawbacks associated with them. The healing process may take weeks, and pain and discomfort continue throughout.

Another common rectal disorder is hemorrhoids, which may be treated by applying a medicament to the anal region. JP 2003-062007 discloses a device capable of prolonging the effect of a hemorrhoids treating medicine. The device comprises a rod shaped core surrounded by a sheet member made of an absorptive material. An anus inserting member comprising a medicament is formed in a protruded shape on the surface of the sheet member. The device is inserted to the patient's anus and adhered externally to his buttocks by an adhesive materiel situated on the surface of the outer flaps of the sheet member. The core member is made of a thermally softening material to make it more comfortable for the individual to engage in daily activities. However, the core member still maintains the protruding shape of the device within the rectum, which, due to the pressing on the rectal wall will give rise to the urge to perform a bowel movement, which, in turn, will force the device out of the anus. Additionally, studies have shown that when the lesion is in a moist environment and includes internal fluids and secretions with the active ingredient (the medicament), the pace of healing process is increased. In JP 2003-062007, an absorptive outer layer is provided, such that internal fluids and secretions are not accessible to the device for combination with the medicament to assist in the healing process.

Independent studies regarding the importance of a moist environment for wound epithelization were conducted and presented as early as the 1960's. The data included both experimental (8) and clinical (9,10) conditions. Tests performed on guinea pigs showed that complete epithelization of the moist wound occurred on day 3, whereas in an exposed (open) superficial wound, complete epithelization occurred only by day 6 or 7. The rates of epithelial cell migration in the moist wound and in the exposed superficial wound was 21 μm/hour and 7 μm/hour, respectively (11).

It is understood today that occlusive dressing promotes wound cleansing, vascularization and restoration of the dermis and epidermis.(12) One of the first clinical trials using dressing Op-Site™ in donor sites of 53 patients showed that the overall healing time was faster than that obtained by conventional methods. The main finding was the complete absence of pain or tenderness in Op-Site™ covered wounds (13).

It is therefore an object of the present invention to provide a method and device for application to a body cavity, with or without an active agent.

It is another object of the present invention to provide a method and device for the prolonged delivery of an active agent to a body cavity, for local or systemic delivery.

It is an additional object of the present invention to provide a method and device for the prolonged rectal delivery of an active agent.

It is an additional object of the present invention to provide a method and device for the delivery of an active agent in a moist environment.

It is an additional object of the present invention to provide a method and device for treatment of a rectal disorder, which overcomes the drawbacks associated with the prior art techniques.

It is an additional object of the present invention to provide a method and device for treatment of a rectal disorder, having minimal negative side affects.

It is an additional object of the present invention to provide a method and device for the treatment of anal fissure.

Additional objects and advantages of the present invention shall become apparent as the description proceeds.

### SUMMARY OF THE INVENTION

In a first embodiment, the present invention relates to a device for application to a body cavity, wherein said device is insertable into said cavity of a subject in need, comprising:
a. a non-absorbable, flexible tube comprising an elongated shape;
b. a removable core element situated within said tube; and
c. a retention mechanism for maintaining said device within said cavity.

In a second embodiment, the present invention further relates to a device for the prolonged delivery of an active agent to a body cavity, wherein said device is insertable into said cavity of a subject in need, and comprises:
a. a non-absorbable, flexible tube comprising an elongated shape having;
b. a removable core element situated within said tube;
c. an active agent; and,
d. a retention mechanism for maintaining said device within said cavity.

The tube is preferably liquid impermeable or at least liquid semi-permeable.

The tube may be naturally-occurring or synthetic, for example, a polymer, selected from the group consisting of
a. cellulose;
b. a polysaccharide, such as chitosan;
c. polylactic acid);
d. poly(glycolic acid);
e. silicone, polyamide;
f. polypeptide; and,
g. polyolefin.

The tube is essentially cylindrical.

Preferably, the core element may be chosen from the group consisting of
a. a rigid material;
b. a gaseous substance.

The tube further comprises an open end through which the core element is removed from said tube.

According to one aspect, the rigid material is maintained in an essentially solid state outside of the body, and in converted to a liquid state when situated in said body.

Preferably, the active agent may be any one of the group consisting of:
a. analgesic;
b. anthelmintic;
c. antibacterial;
d. antiviral;
e. antiprotozoal;
f. antidiarrheal;
g. antihemorrhoidal;
h. hemostatic;
i. anti-inflammatory;
j. a diagnostic aid;
k. a hair remover;
l. a disinfectant;
m. antifungal;
n. an alkylating agent;
o. a muscle relaxant;
p. an antacid;
q. a sedative;
r. mucolytic;
s. contrast media;
t. an antidote;
u. a metabolidendocrinal preparation; and
v. an anti-cancer agent.

Preferably, the active agent is situated at any one of the following locations:
a. the external surface of the tube;
b. the internal surface of said tube; and,
c. within a network of pores of which said tube is comprised.

Alternatively, the active agent is an integral component of the tube.

The retention mechanism comprises any one of the group consisting of
a. an external portion;
b. an internal portion; and,
c. a combination thereof.

The external portion comprises at least one wing extending from the open end of the tube, and wherein at least one surface of said wing comprises adhesive material for adhering said wing to the subject in need.

The internal portion comprises at least one flap portion situated longitudinally along the elongated tube, wherein said flap is an integral portion of said tube, and wherein the outer surface of said flap comprises adhesive material for adhering said outer surface to the internal wall of the cavity of the subject in need. The inner surface of the flap comprises adhesive material for adhering said inner surface to the internal wall of the cavity of the subject in need.

The internal portion comprises an inflatable object situated within the tube, wherein when inflated, said object comprises dimensions that are larger than the maximum diameter of the opening of the cavity. The object may be selected from the group consisting of:
a. an inflatable ring; and,
b. inflatable arms.

Optionally the device may be biodegradable, or alternatively, non-biodegradable

The body cavity may be chosen from the group consisting of:
a. rectum;
b. large intestine;
c. small intestine;
d. esophagus;
e. stomach;
f. trachea;
g. bronchus;
h. outer ear canal;
i. inner ear canal;
j. nasal canal;
k. air sinuses;
l. vagina;
m. cervix;
n. uterus;
o. fallopian tubes;
p. urethra (including prostate);
q. bladder; and,
r. intra-articular cavity.

The device is capable of delivery for any one of the group consisting of
a. local treatment; and,
b. systemic treatment.

The present invention further relates to a method of delivering an active agent to a body cavity for a prolonged period, said method comprising:
a. providing a device comprising;
   i. a non-absorbable, flexible tube comprising an elongated shape;
   ii. a removable core element situated within said tube;
   iii. an active agent; and,
   iv. a retention mechanism for maintaining said device within said cavity.
b. inserting said device into said cavity; and,
c. removing said core element from said tube, thereby allowing said tube to collapse within said cavity.

### BRIEF DESCRIPTION OF THE DRAWINGS

In the drawings:

FIG. 1 illustrates a schematic perspective view of a preferred embodiment of the present invention.

FIG. 2 illustrates a longitudinal cross-sectional view taken along A-A of FIG. 1.

FIGS. 3a and 3b illustrate a portion of the device of the present invention disposed within the rectum of a person in need, in an initial position (FIG. 3a) and after an outwardly directed force is applied (FIG. 3b).

FIGS. 4a and 4b illustrate the device of the present invention, wherein the core element is partially removed therefrom (FIG. 4a), and in a collapsed position after the core element is removed (FIG. 4b).

### DETAILED DESCRIPTION OF THE PREFERRED EMBODIMENTS

The present invention relates in general to a device and method for application to a body cavity, and more specifically to a device and method for the prolonged delivery of an active agent to a body cavity. As an illustrative, but non-limitative, example of the apparatus of the invention, embodiments of the device for prolonged rectal delivery of the active agent, particularly for anal fissure, will be described hereinbelow.

Anal fissure (AF) is a common rectal disorder that can affect men and women, both young and old. Treatments for AF range from a warm bath to a surgical procedure, depending on the severity of the affliction, as described herein above. While some treatments have a better success rate than others, all have negative side affects in one form or another, including incontinence and relapse. The present invention provides a device for prolonging the rectal delivery of an active agent, in which the healing process is hastened, and negative side affects involved in AF treatment are minimized.

Although the present invention is described herein in terms of treatment of AF, it is understood that a wide variety of rectal disorders are treatable with the device of the present invention. Some of those disorders include hemorrhoids, perianal abscess, perianal fistula, inflammatory bowel disease, gastrointestinal polypoid disease (Peutz-Jaeger Syndrome) and juvenile polypoids, among others. More generally, however, the present invention relates to the prolonged rectal delivery of any active agent, for local and/or systemic delivery, preferably in a moist environment. Systemic delivery may include delivery of the active agent to the colon, etc.

Moreover, it is understood that the present invention may be utilized in the prolonged delivery of an active agent to other cavities, such as the large intestine, small intestine, esophagus, stomach, trachea, bronchus, outer ear canal, inner ear canal, nasal canal, air sinuses, vagina, cervix, uterus, fallopian tubes, urethra (including prostate), bladder, infra-articular cavity, etc., although embodiments of the present invention may vary slightly according to the particular body cavity to which the device is inserted. It is understood that a surgical procedure is required for inserting the present invention into one or more of the above-mentioned body cavities.

A preferred embodiment of the device of the present invention, shown in FIG. 1 and designated generally by the numeral (100), comprises a flexible hollow tube (110) comprising an open end. (111) and a closed end (112), and a core element (120) situated within tube (110), for maintaining the elongated cylindrical shape of tube (100).

Tube (100) is essentially cylindrical in shape when core element (120) is situated therein, however, it is understood that the general shape and structure of tube (100) is not limited to that shown in the figures herein, and may vary accordingly.

Device (100) is insertable to the rectum of a subject in need, and comprises a retention mechanism for maintaining device (100) within the rectum, following insertion therein. In the preferred embodiment, the retention mechanism comprises both internal and external portions, however, in other embodiments, only one of the portions may be present.

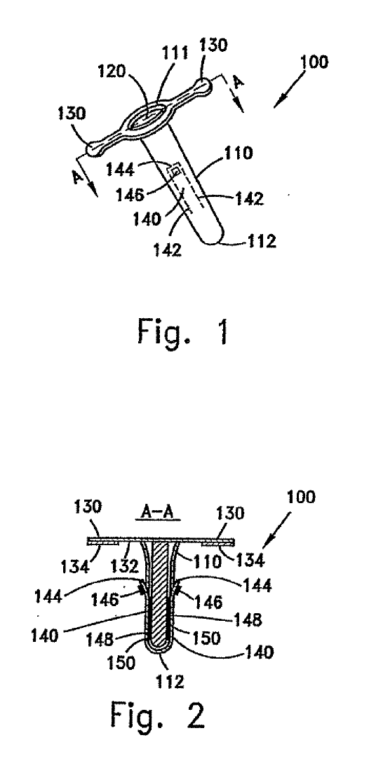

FIG. 2 shows a longitudinal cross-sectional view of device (100) taken along A-A of FIG. 1. Referring to FIGS. 1 and 2, the external portion of the retention mechanism comprises wings (130), which outwardly extend from the open end (111) of tube (110). Wings (130) are preferably integral extensions from the open end (111) of tube (110). According to the preferred embodiment shown in the figures, device (100) comprises two wings, (130), each extending in opposing directions from each other, from open end (111) of tube (110). Alternatively, additional wings may be present, or alternatively, the wing may comprise a continuous sheet extending in all directions from the open end (111) of tube (110). The lower surface (132) of wings (130) comprise adhesive material (134). When the closed end (112) of device (100) is inserted to the rectum, wings (130) are adhered to the skin of the buttocks via adhesive material (134). Thus, if internal pressure is applied to device (100), device (100) is prevented from exiting the rectum.

Still referring to FIGS. 1 and 2, the internal portion of the retention mechanism comprises at least one flap portion (140). Device (100) of the present invention comprises four flaps (140). Flaps (140) are perforated elongated portions of tube (110) situated longitudinally thereon (110). Side walls (142) and top edge (144) of flap are peelable downward, in the direction of closed end (112) of tube (110), as described herein below. Adhesive material (146), e.g. glue, or any bonding substance, is situated near the upper portion of flap (140), for adhesion to the rectal wall upon insertion therein, as described herein below. It may be desirable for adhesive material (146) to extend along the length of the external surface of flap (140). Additional adhesive material (148) is situated along the inner surface (150) of flaps (140) for providing adhesion to the rectal wall when an outwardly directed force is applied, as described herein below.

Top edge (144) of flaps (140) are shown in FIG. 2 slightly peeled for illustrative purposes. Typically, tube (110) is provided initially as having an essentially continuous outer surface. However, in some cases, it may be preferable to provide flaps (140) in the slightly peeled configuration, as shown.

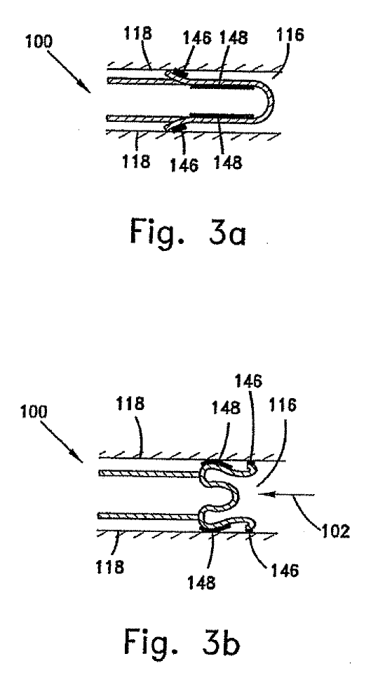

FIG. 3a shows cross-section A-A of device (100) disposed within a rectum (116). Core element (120) and wings (130) are not shown, for purposes of clarity. As seen in the figure, upon insertion, adhesive material (146) naturally adheres to rectum wall (118). As shown in FIG. 3b, if internal pressure builds up such that device (100) is forced in the direction of anal orifice, as indicated by arrow (102), flaps (140) will peel along the perforations. If enough force is applied, flaps (140) will flip over, and adhesive material (148) will adhere to rectal wall (118). Thus, in a preferred embodiment, device (100) is adhered to the patient internally at two locations, i.e. at the locations of adhesive material (146) and (148), and externally, at the location of adhesive material (134).

According to a first aspect of the present invention, core element (120), (FIGS. 1 and 2) comprises a rigid material. Preferably, the rigid material is any hard polymeric material, for example polyolefin or polypropylene. After thrusting device (100) into the rectum, core element (120) is removed. A string, tab or other protruding object is joined to the upper surface of core element (120) at the open end of tube (110). The protruding object is pulled outward, leaving tube (110) within the rectum, as described herein below. Preferably, core element (120) is made of, or at least coated with a non-stick material such as Teflon™ to allow core element (120) to be removed from tube (110) without adhesive material (148) adhering thereto.

According to a second aspect of the present invention, core element (120) is made of a material, which is maintained in an essentially solid state when outside of the body, and is converted to a liquid state when situated within the body. For example core element (120) may be in an essentially solid state when subject to at least ambient temperature, and when subject to higher temperatures, particularly body temperature, is transformed to a liquid state. Shortly after device (100) is inserted to the rectum, core element (120) is converted to a liquid state. In a preferred embodiment, the open end (111) of tube (110) is provided with an absorptive sponge-like material for absorbing core element (120) as it melts (not shown in the figures). According the second aspect, core element (120) is a solidified gel or liquid, or a compressed powder. For example, core element (120) may at least partially comprise saccharides, fillers and other water soluble materials, and manufactured by techniques known in the art.

According to a third aspect of the present invention, the core element is comprised of a gaseous substance and sealed inside a thin housing within tube (110). The gaseous substance is preferably air or any inert gas such as nitrogen. The gaseous substance is pressurized to provide tube (110) with its elongated cylindrical shape. Upon insertion to the rectum, the gaseous substance that comprises core element (120) is released from open end (111) of tube, for instance, by rupturing of the sealed housing via squeezing of tube (110). Alternatively, the gaseous substance may be provided within the tube, and the tube may be sealed at both ends (not shown in the figures). Upon squeezing of the tube, one end of the tube, preferably the end disposed toward the outside of the body, is ruptured, thereby releasing the gaseous substance.

According to any one of the three aspects as described herein above, although preferably with respect to the third aspect, the internal retention mechanism may comprise an object, such as a ring having a diameter larger than the anal orifice, which is situated within tube (110), for preventing tube (110) from exiting the rectum. Alternatively, upon squeezing tube (110), the gaseous substance may enter at least one inflatable member (e.g. an arm, a ring, etc.), thereby causing the member to expand, thereby preventing the removal of tube (110) from the rectum.

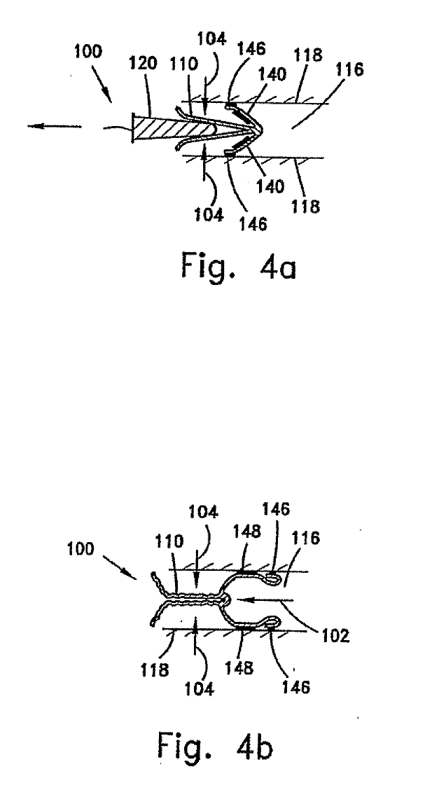

FIG. 4a shows cross-section A-A of device (100) (wings (130) not shown, for clarity) inserted to rectum (116), and core element (120) is partially removed from tube (110). As shown in the figure, when core element (120) is removed from tube (110), tube (110) looses its original cylindrical shape and collapses inward, as indicated by arrows (104). As tube (110) collapses, flaps (140), which are adhered to rectal wall (118) by adhesive material (146) are peeled from the body of tube (110).

After core element (120) is completely removed from tube (110), as shown in FIG. 4b, tube (110) essentially completely collapses; however, adhesion to rectal wall (118) is maintained by adhesive material (146). When internal pressure forces tube (110) in the direction of the anal orifice, as indicated by arrow (102), flaps (140) flip over and adhere to rectal wall (118) at adhesive material (148) as described herein above. By allowing tube (110) to collapse within rectum (116), the patient is not urged to perform a bowel movement due to the presence of device (100). Tube (110), and therefore, the active agent, is maintained within rectum (116), thereby allowing the active agent to be released for a prolonged period of time.

It is understood that the description of the removal of core element (120), as described above, relates to the core element (120) as described in its three different aspects above. In particular, the removal of core element (120) includes the removal of the rigid body, melting of the rigid body, or releasing of the gaseous substance, to allow tube (100) to collapse, mutatis rnutandis.

Tube (110) is preferably made of, or coated with, a non-absorptive material having non-stick properties. Tube (110) may be made of any non-toxic flexible polymeric material, which enables safe and smooth insertion of device (100) according to the invention to a body cavity. Such polymeric material may, for example, comprise polysaccharides, such as cellulose or chitosan, polypeptides, such as gelatin, synthetic polymers, such as polylactic acid), poly(glycolic acid), silicone, polyamide, polyolefin, and mixtures and derivatives thereof. Tube (110) may contain other synthetic or natural physiologically acceptable materials, comprising polymers, fibers, tissues, membranes, and foils. In one example, tube (110) is comprised of dialysis tubing. Preferably, tube (110) comprises a network of pores or micro-pores, within which an active agent is impregnated. Alternatively, the active agent may be an integral component of tube (110), for instance, by melting the agent within the material of tube (110), which serves as a matrix for the active agent. The active agent preferably consists of a diluted paste that is affixed to the outer and/or inner surface of tube (110). Tube (110) is preferably liquid impermeable, however, when the active agent is affixed to the inner surface thereof, tube (110) is semi-permeable to allow the active agent to be released to the surrounding area.

It is believed that the device of the invention provides benefit to patients suffering from anal fissure even in the absence of an active agent.

The active agent may be any one of the following: analgesic, anthelmintic, antibacterial, antiviral, antiprotozoal, antidiarrheal, hemostatic, anti-inflammatory, a diagnostic aid, a hair remover, a disinfectant, antifungal, an alkylating agent, a muscle relaxant, an antacid, a sedative, mucolytic, contrast media, an antidote, or any miscellaneous preparation, such as metabolic/endocrinal preparation or any anti-cancer agent. Tube (110) may be non-biodegradable, and thereby, removable, for example, when the patient performs a bowel movement. Tube (110) may alternatively be biodegradable.

### Example I

### Treatment of Anal Fissure

Three patients suffering from anal fissure were treated with an internal occlusive dressing (hereinafter “device”) of the invention coated with tissue growth factor (TGF), fibrin producing agent and homeopathic preparations and natural oils selected from the group consisting of tea tree oil, calendula oil, propolis extract, and hammamelis extract. Case studies are reported below.

### Case Study #1

A 33 year old female presented shortly after giving birth, complaining of recurrent episodes of painful and bleeding bowel movement due to anal fissure and constipation. The patient had previously been treated with a wide spectrum of treatments including steroid ointments, nitroglycerin spray, warn “Zitz” baths and special diets. The patient also sought help from a range of alternative medical treatments. When first examined, the patient appeared very anxious, panicky, and in pain. Anal examination revealed anal fissure 15 mm. long, of moderate depth. The remainder of the physical examination was good. The patient was treated with an internal occlusive dressing device of the invention that was fixed to the tissue on its external side with Micropore™ paper surgical tape from 3M, and on its internal side with acetyl cellulose. The patient was instructed in the use of the device, which was inserted in the anal canal and left in place until the next bowel movement when it was displaced and flushed from the body. Within 10 minutes of application of the device, while still in the clinic, the patient reported that the pain had disappeared. Upon examination three days late, the area of the previously seen anal fissure appeared to be fully epithelialized. The patient was instructed to continue treatment with the device for another two days. The next year was without complaints. The following year the patient had another episode of pain that seemed to arise from the same cause, i.e., anal fissure. She was instructed to begin treatment immediately with devices that were supplied. Rapid improvement was seen once again. Patient stopped treatment after three days. Since 2002, there were no other complaints.

### Case Study #2

A 66 year old presented, overall in good health but with a history of constipation for the past 30 years that resulted in hemorrhoids, pain and bleeding. He underwent standard treatment including a special diet for loosening the stool and anti-hemorrhoid ointments with Zitz baths. He presented to the clinic with complaints of strong pains of a different nature that continued even after bowel movements. Examination revealed external engorged hemorrhoid of moderate depth and anal fissure 20 mm long. The patient was treated in the clinic with an internal occlusive dressing device of the invention and instructed to continue application of the device for 7 days. The patient reported meaningful relief after several hours. After 24 hours, the patient reported spontaneous drainage of the engorged hemorrhoids, with simultaneous and complete disappearance of pain. The patient was instructed to continue the treatment for another 5 days. Continued follow-up for six years indicates one incident of engorged hemorrhoids, which was successful treated with the device of the invention, and no further recurrence of anal fissure.

### Case Study #3

A 29 year old female in good health, 2 months post-partum, presented to the clinic. Since giving birth, she suffered serious peri-anal pains thought to be related to peri-anal sutures following delivery. She complained of constipation for the past two months, stating she was nursing and not drinking sufficiently. Examination revealed a deep anal fissure of 12 mm long in the ventral position (six o′ clock position). Besides being anxious and frightened, remainder of the examination revealed patient was in overall good health. Rectal checking was not completed due to the pains. Patient did not cooperate with treatment initially. On the fourth day, using analgesic gel, patient succeeded in inserting the internal occlusive dressing device of the invention. Pain relief was achieved within a day. Patient completed a 7-day course of treatment. No follow-up is available as the patient relocated.

These case studies support the results obtained with the occlusive dressing device of the invention and show that the device of the invention is effective in treating and healing anal fissure and hemorrhoids.

While some embodiments of the invention have been described by way of illustration, it will be apparent that the invention can be carried into practice with many modifications, variations and adaptations, and with the use of numerous equivalents or alternative solutions that are within the scope of persons skilled in the art, without departing from the spirit of the invention or exceeding the scope of the claims.

### BIBLIOGRAPHY

1. Schoeten W R, Briel J W, Auwerda J J A, et al. Anal fissure: new concepts in pathogenesis and treatment. Scand J. Gastorenterol 1996; 31 suppl. 218: 78-81
2. Lund J N, Scholefield J H. Aetiology and treatment of anal fissure. Br. J. Surg. 1996; 83: 1335-1344
3. Klosterhalfen B., Vogel P., Rixen H. et al. Topography of the inferior rectal artery: a possible cause of chronic primary anal fissure. Dis. Colon Rectum 1989; 32: 43-52.
4. Lund J N, Scholefield J H. A randomized, prospective, double blind, placebo controlled trial of glyceryl trinitrate ointment of anal fissure. Lancet 1997; 349: 11-14
5. Regadas F S P, Batista L K de O, Albuquerque J L A et al. Pharmacological study of the internal anal sphincter in patients with chronic anal fissure. Br J. Surg. 1993; 80: 799-801
6. Bacher H., Mischinger H J, Werkgartner G., et al. Local nitroglycerin for treatment of anal fissure: an alternative to lateral internal sphincterotomy? Dis Colon Rectum 1997; 40: 849-854
7. Hananel N., Gordon P H. Lateral internal sphincterotomy for fissure in ano-revisited. Dis Colon Rectum 1997; 40: 597-602
8. Winter G D. Formation of scab and the rate of epithelization of superficial wounds in the skin of the young domestic pig. Nature 1962; 193: 293-294.
9. Breitman R S. Outpatient wounds treatment with polychlorvinyl film dressings. Orthoped Traumatol (Moscow) 1963; No. 2: 67-68 (RUS)
10. Hinman C D & Maibach H. Effect of air exposure and occlusion on experimental human skin wound. Nature 1963; 200: 377-378
11. Winter G D. Movement of epidermal cells over the wound surface. In Montagna W & Billingham R E (eds) “Wound Healing”. Proceeding of the Brown University symposium on the Biology of Skin, 1963. Pergamon Press, Oxford et al., 1964, pp. 113-127.
12. Carver N. & Leigh I M. Synthetic dressing. Int. J. Dermatol 1992; 31: 10-18
13. James J R & Watson A C H. The use of Op Site, a vapor permeable dressing, on skin graft donor sites. Br J. Plast. Surg. 1975; 28: 107-110

1. A device for application to a body cavity, wherein said device is insertable into said cavity of a subject in need, comprising:
a. a non-absorbable, flexible tube comprising an elongated shape;
b. a removable core element situated within said tube; and
c. a retention mechanism for maintaining said device within said cavity.

2. A device for the prolonged delivery of an active agent to a body cavity, wherein said device is insertable into said cavity of a subject in need, and comprises:
a. a non-absorbable, flexible tube comprising an elongated shape;
b. a removable core element situated within said tube;
c. an active agent; and,
d. a retention mechanism for maintaining said device within said cavity.

3. A device according to claim 2, wherein the tube is liquid impermeable.

4. A device according to claim 2, wherein the tube is liquid semi-permeable.

5. A device according to claim 2, wherein said tube comprises a polymer selected from the group consisting of:
a. cellulose;
b. chitosan;
c. polylactic acid);
d. poly(glycolic acid);
e. silicone, polyimide;
f. polypeptide; and,
g. polyolefin.

6. A device according to claim 2, wherein the tube is essentially cylindrical.

7. A device according to claim 2, wherein the core element may be chosen from the group consisting of
a. a rigid material;
b. a gaseous substance.

8. A device according to claim 7, wherein the tube further comprises an open end through which the core element is removed from said tube.

9. A device according to claim 7, wherein the rigid material is maintained in an essentially solid state outside of the body, and in converted to a liquid state when situated within said body.

10. A device according to claim 2, wherein the active agent may be any one of the group consisting of
a. analgesic;
b. anthelmintic;
c. antibacterial;
d. antiviral;
e. antiprotozoal;
f. antidiarrheal;
g. antihemorrhoidal;
h. hemostatic;
i. anti-inflammatory;
j. a diagnostic aid;
k. a hair remover;
l. a disinfectant;
m. antifungal;
n. an alkylating agent;
o. a muscle relaxant;
p. an antacid;
q. a sedative;
r. mucolytic;
s. contrast media;
t. an antidote;
u. a metabolic/endocrinal preparation; and
v. an anti-cancer agent.

11. A device according to claim 2, wherein the active agent is situated at any one of the following locations:
a. the external surface of the tube;
b. the internal surface of said tube; and,
c. within a network of pores of which said tube is comprised.

12. A device according to claim 2, wherein the active agent is an integral component of the tube.

13. A device according to claim 8, wherein the retention mechanism comprises any one of the group consisting of
a. an external portion;
b. an internal portion; and,
c. a combination thereof.

14. A device according to claim 13, wherein the external portion comprises at least one wing extending from the open end of the tube, and wherein at least one surface of said wing comprises adhesive material for adhering said wing to the subject in need.

15. A device according to claim 13, wherein the internal portion comprises at least one flap portion situated longitudinally along the elongated tube, wherein said flap is an integral portion of said tube, and wherein the outer surface of said flap comprises adhesive material for adhering said outer surface to the internal wall of the cavity of the subject in need.

16. A device according to claim 15, wherein the inner surface of the flap comprises adhesive material.

17. A device according to claim 13, wherein the internal portion comprises an inflatable object situated within the tube, wherein when inflated, said object comprises dimensions that are larger than the maximum diameter of the opening of the cavity.

18. A device according to claim 17, wherein the object may be selected from the group consisting of:
a. an inflatable ring; and,
b. inflatable arms.

19. A device according to claim 2, wherein said device is chosen from any one of the group consisting of
a. biodegradable; and,
b. non-biodegradable

20. A device according to claim 2, wherein the body cavity may be chosen from the group consisting of:
a rectum;
b. large intestine;
c. small intestine;
d. esophagus;
e. stomach;
f. trachea;
g. bronchus;
h. outer ear canal;
i. inner ear canal;
j. nasal canal;
k. air sinuses;
l. vagina;
m. cervix;
n. uterus;
o. fallopian tubes;
p. urethra (including prostate);
q. bladder; and,
r. intra-articular cavity.

21. A device according to claim 2, wherein said device is capable of delivery for any one of the group consisting of
a. local treatment; and,
b. systemic treatment.

22. A method of delivering an active agent to a body cavity for a prolonged period, said method comprising:
a. providing a device comprising;
i. a non-absorbable, flexible tube comprising an elongated shape;
ii. a removable core element situated within said tube;
iii. an active agent; and,
iv. a retention mechanism for maintaining said device within said cavity.
b. inserting said device into said cavity; and,
c. removing said core element from said tube, thereby allowing said tube to collapse within said cavity.

23. A method for the local treatment of anal fissure in a subject in need thereof, comprising at least one administration to said subject of a device as defined in claim 2.
